(19) United States
(12) Patent Application Publication
(10) Pub. No.: US 2019/0076456 A1
(43) Pub. Date: Mar. 14, 2019

(54) COMPOSITIONS AND METHODS FOR LIVER DISEASES

(71) Applicant: Stephen McNERLIN, Grand Cayman (KY)

(72) Inventor: Stephen McNERLIN, Grand Cayman (KY)

(21) Appl. No.: 15/702,528

(22) Filed: Sep. 12, 2017

(51) Int. Cl. A61K 31/7084; A61K 9/00

(52) U.S. Cl. CPC A61K 31/7084; A61K 9/0073; A61K 9/0019; A61K 9/0078

(57) ABSTRACT

Embodiments of the present invention disclose a composition comprising nicotinamide adenine dinucleotide (NAD+), wherein the NAD+ is in an amount effective for a liver disorder in a mammal. Methods of making and using the invention composition are also disclosed.

### FIELD OF THE INVENTION

The present invention provides a novel formulation for reducing, ameliorating or treating liver diseases or related conditions and methods of making and using the same.

### BACKGROUND OF THE INVENTION

Liver diseases are common among all age groups of both sexes. Many factors can cause a human being to suffer from liver diseases, such factors including, e.g., viruses for hepatitis A, B, and C; excessive eating or over-nutrition for fatty liver, and Therefore, there is a continuing need for compositions and methods to treat or ameliorate a condition associated with ageing.

The embodiments described below address the above identified issues and need.

### SUMMARY OF THE INVENTION

In one aspect of the present invention, it is provided a composition for treating or ameliorating a liver disease or a related condition, comprising an effective amount of nicotinamide adenine dinucleotide (NAD+).

In some embodiments of the invention composition, optionally in combination with any of the various embodiments disclosed herein, the composition is in a dosage form or a dosing regimen for the liver disease or related condition.

In some embodiments of the invention composition, optionally in combination with any of the various embodiments disclosed herein, the dosage form comprises a dosing applicator.

In some embodiments of the invention composition, optionally in combination with any of the various embodiments disclosed herein, the liver disease is a chronic alcoholic liver disease.

In some embodiments of the invention composition, optionally in combination with any of the various embodiments disclosed herein, the liver disease is a non-alcoholic liver disease.

In some embodiments of the invention composition, optionally in combination with any of the various embodiments disclosed herein, the liver disease is fatty liver.

In some embodiments of the invention composition, optionally in combination with any of the various embodiments disclosed herein, the related condition is alcohol hangover.

In some embodiments of the invention composition, optionally in combination with any of the various embodiments disclosed herein, the composition further comprises a carrier for pulmonary delivery.

In some embodiments of the invention composition, optionally in combination with any of the various embodiments disclosed herein, the pulmonary delivery is by nebulizer.

In some embodiments of the invention composition, optionally in combination with any of the various embodiments disclosed herein, the pulmonary delivery is by nasal spray or nasal inhaling.

In some embodiments of the invention composition, optionally in combination with any of the various embodiments disclosed herein, the pulmonary delivery is by an inhaler.

In some embodiments of the invention composition, optionally in combination with any of the various embodiments disclosed herein, the composition is in a subcutaneous pellet form of injections for long term delivery.

In a second aspect of the present invention, it is provided a method for treating or ameliorating a liver disease or a related condition in a mammal, comprising administering to said mammal a comprising an effective amount of nicotinamide adenine dinucleotide (NAD+) in a dosage form or a dosing regimen for the liver disease or related condition.

In some embodiments of the invention method, optionally in combination with any of the various embodiments disclosed herein, the dosage form comprises a dosing applicator.

In some embodiments of the invention method, optionally in combination with any of the various embodiments disclosed herein, the liver disease is a chronic alcoholic liver disease.

In some embodiments of the invention method, optionally in combination with any of the various embodiments disclosed herein, the liver disease is a non-alcoholic liver disease.

In some embodiments of the invention method, optionally in combination with any of the various embodiments disclosed herein, the liver disease is fatty liver.

In some embodiments of the invention method, optionally in combination with any of the various embodiments disclosed herein, the related condition is alcohol hangover.

In some embodiments of the invention method, optionally in combination with any of the various embodiments disclosed herein, the composition further comprises a carrier for pulmonary delivery.

In some embodiments of the invention method, optionally in combination with any of the various embodiments disclosed herein, the pulmonary delivery is by nebulizer.

In some embodiments of the invention method, optionally in combination with any of the various embodiments disclosed herein, the pulmonary delivery is by nasal spray or nasal inhaling.

In some embodiments of the invention method, optionally in combination with any of the various embodiments disclosed herein, the pulmonary delivery is by an inhaler.

In some embodiments of the invention method, optionally in combination with any of the various embodiments disclosed herein, the pulmonary delivery is delivering a subcutaneous pellet form of injections for long term delivery.

In some embodiments of the invention method, optionally in combination with any of the various embodiments disclosed herein, the mammal is an animal or a human being.

In a third aspect of the present invention, it is provided a method of fabricating a composition, comprising providing nicotinamide adenine dinucleotide (NAD+) in an effective amount for a liver disease or related condition and forming a composition.

In some embodiments of the invention method, optionally in combination with any of the various embodiments disclosed herein, the composition is in a dosage form or a dosing regimen for the liver disease or related condition.

In some embodiments of the invention method, optionally in combination with any of the various embodiments disclosed herein, the dosage form comprises a dosing applicator.

In some embodiments of the invention method, optionally in combination with any of the various embodiments disclosed herein, the liver disease is a chronic alcoholic liver disease.

In some embodiments of the invention method, optionally in combination with any of the various embodiments disclosed herein, the liver disease is a non-alcoholic liver disease.

In some embodiments of the invention method, optionally in combination with any of the various embodiments disclosed herein, the liver disease is fatty liver.

In some embodiments of the invention method, optionally in combination with any of the various embodiments disclosed herein, the related condition is alcohol hangover.

In some embodiments of the invention method, optionally in combination with any of the various embodiments disclosed herein, the composition further comprises a carrier for pulmonary delivery.

In some embodiments of the invention method, optionally in combination with any of the various embodiments disclosed herein, the pulmonary delivery is by nebulizer.

In some embodiments of the invention method, optionally in combination with any of the various embodiments disclosed herein, the pulmonary delivery is by nasal spray or nasal inhaling.

In some embodiments of the invention method, optionally in combination with any of the various embodiments disclosed herein, the pulmonary delivery is by an inhaler.

In some embodiments of the invention method, optionally in combination with any of the various embodiments disclosed herein, the pulmonary delivery is delivering a subcutaneous pellet form of injections for long term delivery.

### DETAILED DESCRIPTION OF THE INVENTION

### Definitions

As used herein, the terms “administering” or “administration” of an agent, drug, or peptide to a subject includes any route of introducing or delivering to a subject a compound to perform its intended function. The administering or administration can be carried out by any suitable route, including orally, intranasally, parenterally (intravenously, intramuscularly, intraperitoneally, or subcutaneously), rectally, or topically. Administering or administration includes self-administration and the administration by another.

As used herein, the term “rapid release” shall mean the release dosage forms for which ≥80% (e.g., about 80%, 85%, 90%, 95%, 98% or 99%) of labelled amount releases within 30 min. Conversely, the term “sustained release” shall mean the release dosage forms for which ≥80% (e.g., about 80%, 85%, 90%, 95%, 98% or 99%) of labelled amount releases over a period ≥30 minutes, e.g., about 45 minutes, 60 minutes, 90 minutes, 120 minutes, 2 hours, 4 hours, 6 hours, 8 hours, 12 hours, 24 hours, 2 days, 3 days, 4 days, 5 days, 6 days, 7 days, or beyond. The term “rapid release” is used interchangeably with the term “rapid delivery”, “fast release”, “fast delivery”, “immediate release”, or “immediate delivery.” The term “sustained release” is used interchangeably with the term “sustained delivery”, “long term release”, “long term delivery”, “delayed release”, or “delayed delivery.”

Pulmonary delivery of drug has become an attractive target as the lung is capable of absorbing pharmaceuticals either for local deposition or for systemic delivery. The pulmonary route as a non-invasive administration is suitable for systemic and local delivery of therapeutic agents, because the high permeability and large absorptive surface area of lungs, and good blood supply. As used herein, the term “pulmonary delivery” includes drug delivery via nasal spray, nasal inhaling, an oral inhaler, or any other drug delivery techniques capable of causing a drug to be delivered to the respiratory track and/or lung.

As used herein, the terms “disease,” “disorder,” or “complication” refers to any deviation from a normal state in a subject. In preferred embodiments, the methods and compositions of the present invention are useful in the treatment or management of a liver disease or a related condition.

As used herein, by the term “effective amount,” “amount effective,” “therapeutically effective amount,” or the like, it is meant an amount effective at dosages and for periods of time necessary to achieve the desired result. Generally, such effective amount refers to an amount of NAD+ that is capable of increasing blood level NAD+/NADH ratio to have a therapeutically significant effect on a liver disorder in a mammal subject. Examples of such effective amount range from about 1 μg to about 10 grams (e.g., about 2 μg, 5 μg, 10 μg, 20 μg, 50 μg, 100 μg, 200 μg, 500 μg, 1 mg, 10 mg, 20 mg, 50 mg, 100 mg, 200 mg, 500 mg, or about 1000 mg) per kilogram body weight.

As used herein, a “formulation,” “pharmaceutical formulation” all include a composition comprising at least one of NAD+ or a precursor thereof. Optionally, the “composition,” “pharmaceutical composition” or “therapeutic agent” further comprises pharmaceutically acceptable diluents or carriers.

As used herein, the term “preventing” means causing the clinical symptoms of the disease state not to develop, e.g., inhibiting the onset of disease, in a subject that may be exposed to or predisposed to the disease state, but does not yet experience or display symptoms of the disease state.

As used herein, the term “subject” refers to any animal (e.g., a mammal), including, but not limited to, humans, non-human primates, rodents, and the like, which is to be the recipient of a particular treatment.

As used herein, the terms “treating” or “treatment” or “alleviation” refers to both therapeutic treatment and prophylactic or preventative measures, wherein the object is to prevent or slow down (lessen) the targeted pathologic condition or disorder.

As used herein, the terms “composition” and “formulation” are sometimes used interchangeably.

### Nicotinamide Adenine Dinucleotide

Nicotinamide adenine dinucleotide (NAD) is a coenzyme found in all living cells. The compound is a dinucleotide, because it consists of two nucleotides joined through their phosphate groups. One nucleotide contains an adenine base and the other nicotinamide. Nicotinamide adenine dinucleotide exists in two forms, an oxidized and reduced form abbreviated as NAD+ and NADH respectively, as shown below:

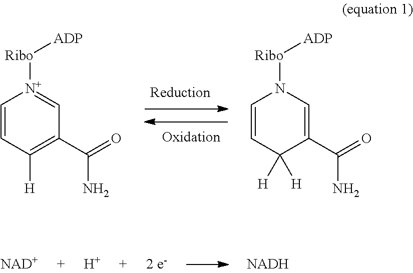

In metabolism, nicotinamide adenine dinucleotide is involved in redox reactions, carrying electrons from one reaction to another. The coenzyme is, therefore, found in two forms in cells: NAD+ is an oxidizing agent—it accepts electrons from other molecules and becomes reduced. This reaction forms NADH, which can then be used as a reducing agent to donate electrons. This electron transfer reactions are the main function of NAD. However, it is also used in other cellular processes, the most notable one being a substrate of enzymes that add or remove chemical groups from proteins, in posttranslational modifications. Because of the importance of these functions, the enzymes involved in NAD metabolism are targets for drug discovery.

In organisms, NAD can be synthesized from simple building-blocks (de novo) from the amino acids tryptophan or aspartic acid. In an alternative route, more complex components of the coenzymes are taken up from food as vitamin niacin. Similar compounds are released by reactions that break down the structure of NAD. These preformed components then pass through a salvage pathway that recycles them back into the active form. Some NAD is also converted into nicotinamide adenine dinucleotide phosphate (NADP); the chemistry of this related coenzyme is similar to that of NAD, but it has different roles in metabolism.

Although NAD+ is written with a superscript plus sign because of the formal charge on a particular nitrogen atom, at physiological pH for the most part it is actually a singly charged anion (charge of minus 1), while NADH is a doubly charged anion.

### Effect of NAD+ on Liver Diseases

Liver disease can be inherited (genetic) or caused by a variety of factors that damage the liver, such as viruses and alcohol use. Obesity is also associated with liver damage. Over time, damage to the liver results in scarring (cirrhosis), which can lead to liver failure, a life-threatening condition.

While the exact mechanism of action of NAD+ on liver diseases requires further investigation, Applicant believes, without wishing to be bound by the theory, that the possible mechanisms of NAD+ on liver diseases are: 1) regulation of SIRT1 by NAD+ and 2) direct and positive involvement of NAD+ in alcohol metabolism.

### Regulation of SIRT1 by NAD+

While the functions of NAD+ are the focus of many studies, it is believed the mechanism of action of NAD+ with respect to the invention disclosed herein is its ability to modulate the level of SIRT1. A decrease of SIRT1 level, in turn, reportedly is linked to many disorders including aging-associated disorders (see, e.g., Ng, F., et al., SIRT1 in the brain—connections with aging-associated disorders and lifespan, in Frontiers in Cellular Neuroscience, March 2015, vol. 9, Article 64 (Review). NAD has been reported to reverse aging in that it enables communication inside cells between the nucleus and mitochondria (https://hms.harvard.edu/news/genetics/new-reversible-cause-aging-12-19-13). A study reported in 2012 there exists a link between oxidative stress and PARP activity, aging, and a decline in NAD+ levels in human tissue (Massudi, H., et al., PLoS One, July 2012, Vol. 7 (7): e42357). NAD's ability to restore cell communication to reverse the aging process is also confirmed in another report (https://www.theguardian.com/science/2013/dec/20/anti-ageing-human-trials).

As used herein, while the term “NAD” is sometimes used instead of NAD+, the reference to NAD in the instant application shall mean the net amount of NAD+ in the NAD is higher than NADH in that the molar ratio of NAD+/NADH in the NAD>1.

### Alcohol Metabolism and NAD+

NAD+ is involved in the interconversion between alcohol and ketones and aldehyde. The following equation shows the conversion between alcohol and aldehyde involving NAD+:

CH₃CH₂OH+NAD⁺→CH₃CHO+NADH+H⁺

See, e.g., Sofer W, Martin PF (1987),“Analysis of alcohol dehydrogenase gene expression in Drosophila”, Annual Review of Genetics. 21: 203-25.

Further, NAD+ oxidizes aldehyde to form an acid and NADH:

Aldehyde+NAD⁺+H₂O→Acid+NADH+H⁺.

It is clear from the equation that the NAD+/NADH ratio is an important factor in the interconversion between alcohol and aldehyde.

### Pharmaceutical Compositions/Formulation

Therefore, in one aspect of the present invention, it is provided a composition for treating or ameliorating a liver disease or a related condition, comprising an effective amount of nicotinamide adenine dinucleotide (NAD+).

In some embodiments of the invention composition, optionally in combination with any of the various embodiments disclosed herein, the composition is in a dosage form or a dosing regimen for the liver disease or related condition.

In some embodiments of the invention composition, optionally in combination with any of the various embodiments disclosed herein, the dosage form comprises a dosing applicator.

In some embodiments of the invention composition, optionally in combination with any of the various embodiments disclosed herein, the liver disease is a chronic alcoholic liver disease.

In some embodiments of the invention composition, optionally in combination with any of the various embodiments disclosed herein, the liver disease is a non-alcoholic liver disease.

In some embodiments of the invention composition, optionally in combination with any of the various embodiments disclosed herein, the liver disease is fatty liver.

In some embodiments of the invention composition, optionally in combination with any of the various embodiments disclosed herein, the related condition is alcohol hangover.

In some embodiments of the invention composition, optionally in combination with any of the various embodiments disclosed herein, the composition further comprises a carrier for pulmonary delivery.

In some embodiments of the invention composition, optionally in combination with any of the various embodiments disclosed herein, the pulmonary delivery is by nebulizer.

In some embodiments of the invention composition, optionally in combination with any of the various embodiments disclosed herein, the pulmonary delivery is by nasal spray or nasal inhaling.

In some embodiments of the invention composition, optionally in combination with any of the various embodiments disclosed herein, the pulmonary delivery is by an inhaler.

In some embodiments of the invention composition, optionally in combination with any of the various embodiments disclosed herein, the composition is a subcutaneous pellet form of injections for long term delivery.

The compositions can be administered alone or in combination with at least one other agent, such as stabilizing compound, which can be administered in any sterile, biocompatible pharmaceutical carrier, including, but not limited to, saline, buffered saline, dextrose, and water. The compositions can be administered to a patient alone, or in combination with other agents, drugs or hormones.

In addition to the active ingredients, these compositions can contain suitable pharmaceutically acceptable carriers comprising excipients and auxiliaries which facilitate processing of the active compounds into preparations which can be used pharmaceutically. Compositions of the invention can be administered by any number of routes including, but not limited to, oral, intravenous, intramuscular, intra-arterial, intramedullary, intrathecal, intraventricular, transdermal, subcutaneous, intraperitoneal, intranasal, parenteral, topical, sublingual, or rectal means. Compositions for oral administration can be formulated using pharmaceutically acceptable carriers well known in the art in dosages suitable for oral administration. Such carriers enable the pharmaceutical compositions to be formulated as tablets, pills, dragees, capsules, liquids, gels, syrups, slurries, suspensions, and the like, for ingestion by the patient.

The composition of invention can be formulated for rapid release or sustained release (AKA long term delivery) formulations. Compositions for oral administration can be formulated using pharmaceutically acceptable carriers well known in the art in dosages suitable for oral administration. Such carriers enable the pharmaceutical compositions to be formulated as tablets, pills, dragees, capsules, liquids, gels, syrups, slurries, suspensions, and the like, for ingestion by the patient. An example of injection formulation is subcutaneous pellet form of injections for long term delivery.

Further details on techniques for formulation and administration can be found in the latest edition of REMINGTON'S PHARMACEUTICAL SCIENCES (Maack Publishing Co., Easton, Pa., which is incorporated herein by reference). After pharmaceutical compositions have been prepared, they can be placed in an appropriate container and labeled for treatment of an indicated condition. Such labeling would include amount, frequency, and method of administration.

It is noted that the compositions and methods disclosed herein may be administered to any subject as defined herein. In one embodiment, the subject is human. In another embodiment, the subject is a pet, e.g., cat, dog, or the like, and in a particular embodiment, is an overweight pet or animal. It is further noted that a corresponding composition, e.g., a pharmaceutical composition, may be provided for use in any method described herein.

All forms of NAD coenzyme are white amorphous powders that are hygroscopic and highly water-soluble. The solids are stable if stored dry and in the dark. Solutions of NAD+ are colorless and stable for about a week at 4° C. and neutral pH, but decompose rapidly in acids or alkalis. Upon decomposition, they form products that are enzyme inhibitors. Various formulations therefore can be made making use of such properties of NAD.

Those skilled in the art will appreciate that numerous delivery mechanisms are available for delivering a therapeutic agent to an area of need. By way of example, the agent may be delivered using a liposome as the delivery vehicle. Preferably, the liposome is stable in the animal into which it has been administered for at least about 30 minutes, more preferably for at least about 1 hour, and even more preferably for at least about 24 hours. A liposome comprises a lipid composition that is capable of targeting a reagent, particularly a polynucleotide, to a particular site in an animal, such as a human.

### Method of Fabrication

In a further aspect of the present invention, it is provided a method of fabricating a composition, comprising providing nicotinamide adenine dinucleotide (NAD+) in an effective amount for a liver disease or related condition and forming a composition.

In some embodiments of the invention method, optionally in combination with any of the various embodiments disclosed herein, the composition is in a dosage form or a dosing regimen for the liver disease or related condition.

In some embodiments of the invention method, optionally in combination with any of the various embodiments disclosed herein, the dosage form comprises a dosing applicator.

In some embodiments of the invention method, optionally in combination with any of the various embodiments disclosed herein, the liver disease is a chronic alcoholic liver disease.

In some embodiments of the invention method, optionally in combination with any of the various embodiments disclosed herein, the liver disease is a non-alcoholic liver disease.

In some embodiments of the invention method, optionally in combination with any of the various embodiments disclosed herein, the liver disease is fatty liver.

In some embodiments of the invention method, optionally in combination with any of the various embodiments disclosed herein, the related condition is alcohol hangover.

In some embodiments of the invention method, optionally in combination with any of the various embodiments disclosed herein, the composition further comprises a carrier for pulmonary delivery.

In some embodiments of the invention method, optionally in combination with any of the various embodiments disclosed herein, the pulmonary delivery is by nebulizer.

In some embodiments of the invention method, optionally in combination with any of the various embodiments disclosed herein, the pulmonary delivery is by nasal spray or nasal inhaling.

In some embodiments of the invention method, optionally in combination with any of the various embodiments disclosed herein, the pulmonary delivery is by an inhaler.

In some embodiments of the invention method, optionally in combination with any of the various embodiments disclosed herein, the pulmonary delivery is delivering a subcutaneous pellet form of injections for long term delivery.

In some embodiments of the invention method, optionally in combination with any of the various embodiments disclosed herein, the mammal is a human being.

### Method of Use

In a further aspect of the present invention, it is provided a method for treating or ameliorating a liver disease or a related condition in a mammal, comprising administering to said mammal a comprising an effective amount of nicotinamide adenine dinucleotide (NAD+) in a dosage form or a dosing regimen for the liver disease or related condition.

In some embodiments of the invention method, optionally in combination with any of the various embodiments disclosed herein, the dosage form comprises a dosing applicator.

In some embodiments of the invention method, optionally in combination with any of the various embodiments disclosed herein, the liver disease is a chronic alcoholic liver disease.

In some embodiments of the invention method, optionally in combination with any of the various embodiments disclosed herein, the liver disease is a non-alcoholic liver disease.

In some embodiments of the invention method, optionally in combination with any of the various embodiments disclosed herein, the liver disease is fatty liver.

In some embodiments of the invention method, optionally in combination with any of the various embodiments disclosed herein, the related condition is alcohol hangover.

In some embodiments of the invention method, optionally in combination with any of the various embodiments disclosed herein, the composition further comprises a carrier for pulmonary delivery.

In some embodiments of the invention method, optionally in combination with any of the various embodiments disclosed herein, the pulmonary delivery is by nebulizer.

In some embodiments of the invention method, optionally in combination with any of the various embodiments disclosed herein, the pulmonary delivery is by nasal spray or nasal inhaling.

In some embodiments of the invention method, optionally in combination with any of the various embodiments disclosed herein, the pulmonary delivery is by an inhaler.

In some embodiments of the invention method, optionally in combination with any of the various embodiments disclosed herein, the pulmonary delivery is delivering a subcutaneous pellet form of injections for long term delivery.

In some embodiments of the invention method, optionally in combination with any of the various embodiments disclosed herein, the mammal is an animal or a human being.

### Liver Disorders

The composition disclosed herein can be used to treat or ameliorate alcoholic or non-alcoholic liver disorders, acute liver diseases or chronic liver diseases. Alternatively, liver disease can be inherited (genetic) or caused by a variety of factors that damage the liver, such as viruses and alcohol use. Obesity is also associated with liver damage. Over time, damage to the liver results in scarring (cirrhosis), which can lead to liver failure, a life-threatening condition.

Examples of liver diseases that can be treated or ameliorated by the composition of invention includes, but are not limited to, fatty liver, cirrhosis, and alcohol hangover.

### Dosage Form

In a further aspect of the present invention, it is provided a kit, comprising:
a formulation comprising a carrier and an effective amount of nicotinamide adenine dinucleotide (NAD) via pulmonary delivery to a mammal subject,
a dosing unit that provides one or more doses of the formulation, each dose providing the effective amount of nicotinamide adenine dinucleotide (NAD) via pulmonary delivery to a subject to treat or ameliorate a liver disorder,
an optional liquid housing unit for housing an optional liquid carrier, and
a dispensing unit for dispensing the formulation in the dosing unit for pulmonary application to a subject in need thereof,
wherein the dosage unit, the optional liquid housing unit, and the dispensing unit are separate, partially joined, or entirely joined forming a single structure.

In some embodiments of the invention kit, optionally in combination with any or all of the various embodiments disclosed herein, the application is nasal administration.

In some embodiments of the invention formulation, optionally in combination with any or all of the various embodiments disclosed herein, the liquid carrier is water.

In some embodiments of the invention kit, optionally in combination with any or all of the various embodiments disclosed herein, the pulmonary delivery is by nebulizer.

In some embodiments of the invention kit, optionally in combination with any or all of the various embodiments disclosed herein, the pulmonary delivery is by nasal spray or nasal inhaling.

In some embodiments of the invention kit, optionally in combination with any or all of the various embodiments disclosed herein, the pulmonary delivery is by nasal spray or nasal inhaling via a vaping device.

In some embodiments of the invention kit, optionally in combination with any or all of the various embodiments disclosed herein, the liver disorder is alcohol hangover.

In some embodiments of the invention kit, optionally in combination with any or all of the various embodiments disclosed herein, the liver disorder is fatty liver or cirrhosis.

In some embodiments of the invention kit, optionally in combination with any or all of the various embodiments disclosed herein, the mammal is a human being.

The following examples illustrate rather than limit the embodiments of the present invention.

### EXAMPLES

### Example 1

A male subject, who is 55 years old and suffers from alcohol hangover was administered by nasal spray a composition of invention (with a dosage of 50 mg NAD+ per administration) three times in a day. Significant improvement of symptoms of alcohol hangover was observed.

While various embodiments of the present invention have been shown and described herein, it will be obvious that such embodiments are provided by way of example only. Numerous variations, changes and substitutions may be made without departing from the invention herein. Accordingly, it is intended that the invention be limited only by the spirit and scope of the appended claims.

The teachings of the references, including patents and patent related documents, cited herein are incorporated herein in their entirety to the extent not inconsistent with the teachings herein.

I claim:

1. A composition for treating or ameliorating a liver disease or a related condition, comprising an effective amount of nicotinamide adenine dinucleotide (NAD+).

2. The composition according to claim 1 in a dosage form or a dosing regimen for the liver disease or related condition.

3. The composition according to claim 2, wherein the dosage form comprises a dosing applicator.

4. The composition according to claim 2, wherein the liver disease is a chronic alcoholic liver disease.

5. The composition according to claim 2, wherein the liver disease is a non-alcoholic liver disease.

6. The composition according to claim 2, wherein the liver disease is fatty liver.

7. The composition according to claim 2, wherein the related condition is alcohol hangover.

8. The composition of claim 2, further comprising a carrier for pulmonary delivery.

9. The composition of claim 8, wherein the pulmonary delivery is by nebulizer.

10. The composition of claim 8, wherein the pulmonary delivery is by nasal spray or nasal inhaling.

11. The composition of claim 8, wherein the pulmonary delivery is by an inhaler.

12. The composition of claim 1, which is in a subcutaneous pellet form of injections for long term delivery.

13. A method for treating or ameliorating a liver disease or a related condition in a mammal, comprising administering to said mammal a comprising an effective amount of nicotinamide adenine dinucleotide (NAD+) in a dosage form or a dosing regimen for the liver disease or related condition.

14. The method according to claim 13, wherein the dosage form comprises a dosing applicator.

15. The method according to claim 13, wherein the liver disease is a chronic alcoholic liver disease.

16. The method according to claim 13, wherein the liver disease is a non-alcoholic liver disease.

17. The method according to claim 13, wherein the liver disease is fatty liver.

18. The method according to claim 13, wherein the related condition is alcohol hangover.

19. The method according to claim 13, wherein the composition further comprises a carrier for pulmonary delivery.

20. The method according to claim 19, wherein the pulmonary delivery is by nebulizer.

21. The method according to claim 19, wherein the pulmonary delivery is by nasal spray or nasal inhaling.

22. The method according to claim 19, wherein the pulmonary delivery is by an inhaler.

23. The method according to claim 13, wherein the composition is in a subcutaneous pellet form of injections for long term delivery.

24. A method of fabricating a composition, comprising providing nicotinamide adenine dinucleotide (NAD+) and forming a composition according to claim 1.
